(19) United States
(12) Patent Application Publication
(10) Pub. No.: US 2026/0365789 A1
(43) Pub. Date: Nov. 19, 2026

(54) REACTIVE MULTI-ARM POLYMERS FOR USE IN FORMING MEDICAL HYDROGELS

(71) Applicant: Boston Scientific Scimed, Inc., Maple Grove, MN (US)

(72) Inventor: Yen-Hao Hsu, Shrewsbury, MA (US)

(73) Assignee: Boston Scientific Scimed, Inc., Maple Grove, MN (US)

(21) Appl. No.: 19/665,516

(22) Filed: May 1, 2026

Related U.S. Application Data
(60) Provisional application No. 63/798,282, filed on May 1, 2025.

(51) Int. Cl. C08G 65/00; A61L 27/18

(52) U.S. Cl. CPC C08G 65/005; A61L 27/18; A61L 2400/06; C08G 2210/00

(57) ABSTRACT

In various aspects of the present disclosure, multi-arm polymers are provided that comprise an iodinated core region and three or more polymer arms extending from the iodinated core region, each polymer arm of the three or more polymer arms comprising a hydrophilic polymer segment and a reactive moiety, wherein the reactive moiety is positioned at an end of each polymer arm and is covalently attached to the hydrophilic polymer segment, and wherein the reactive moiety comprises a C₁-C₁₀ alkyl iodide group or an α,β-unsaturated ketone group. Other aspects of the present disclosure pertain to crosslinked hydrogels formed from such multi-arm polymers, to crosslinkable systems for forming such crosslinked hydrogels, and to methods of treatment using such crosslinked hydrogels.

### CROSS-REFERENCE TO RELATED APPLICATIONS

This application claims the benefit of U.S. Provisional Patent Application Ser. No. 63/798,282 filed on May 1, 2025, the disclosure of which is incorporated herein by reference.

### FIELD

The present disclosure relates to functionalized multi-arm polymers, to crosslinked hydrogels formed from such functionalized multi-arm polymers, to crosslinkable systems for forming such crosslinked hydrogels, and to methods of treatment using such crosslinked hydrogels.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically-used biomaterial in prostate cancer therapy. A further improvement based on this structure is that a portion of the succinimidyl glutarate end groups have been replaced with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups of SpaceOAR® and SpaceOAR Vue® will rapidly react with the trilysine crosslinker in vivo to form a hydrogel. The hydrogel breaks down in-vivo over the course of about 6-9 months. The breakdown occurs primarily through the hydrolysis of the ester linkages in the glutarate groups.

The present disclosure provides implantable hydrogel alternatives to SpaceOAR Vue®, thereby expanding the range of medical applications for the hydrogels.

### SUMMARY

In various aspects of the present disclosure, multi-arm polymers are provided that comprise an iodinated core region and three or more polymer arms extending from the iodinated core region, each polymer arm of the three or more polymer arms comprising a hydrophilic polymer segment and a reactive moiety, wherein the reactive moiety is positioned at an end of each polymer arm and is covalently attached to the hydrophilic polymer segment, and wherein the reactive moiety comprises a C₁-C₁₀ alkyl iodide group or an α,β-unsaturated ketone group.

In some embodiments, the reactive moiety comprises the C₁-C₁₀ alkyl iodide group.

In some embodiments, the reactive moiety comprises the α,β-unsaturated ketone group, such as a vinyl ketone group or an ethynyl ketone group.

In some embodiments, which can be used in conjunction with the above aspects and embodiments, the reactive moiety is covalently linked to the hydrophilic polymer segment through a linkage that comprises an ester group.

In some embodiments, which can be used in conjunction with the above aspects and embodiments, the reactive moiety is covalently linked to the hydrophilic polymer segment through a hydrolytically stable linkage.

In some embodiments, which can be used in conjunction with the above aspects and embodiments, hydrophilic polymer segment comprises one or more monomer residues selected from ethylene oxide, propylene oxide, N-vinyl pyrrolidone and oxazoline monomer residues.

In some embodiments, which can be used in conjunction with the above aspects and embodiments, the iodinated core comprises one or more iodinated aromatic groups.

In some embodiments, which can be used in conjunction with the above aspects and embodiments, the iodinated core region comprises a residue of an iodinated polyhydroxy compound.

In other aspects, the present disclosure pertains to systems that comprise (a) a multi-arm polymer in accordance with any of the above aspects and embodiments and (b) a multifunctional compound comprising a plurality of reactive groups that are reactive with reactive moieties of the multi-arm polymer.

In some embodiments, reactive groups are nucleophilic groups. In some of these embodiments, the nucleophilic groups are primary amine groups or secondary amine groups. In some of these embodiments, the nucleophilic groups are thiol groups.

In additional aspects, the present disclosure pertains to methods of treatment comprising administering to a subject a mixture that comprises (a) a multi-arm polymer in accordance with any of the above aspects and embodiments and (b) a multifunctional compound comprising a plurality of reactive groups that are reactive with reactive moieties of the multi-arm polymer, under conditions such that the multi-arm polymer and the multifunctional compound crosslink after administration to form a hydrogel.

In some embodiments, the nucleophilic groups are selected from thiol groups, primary amine groups and secondary amine groups.

In some embodiments, which can be used in conjunction with the preceding aspects and embodiments, the method comprises administering to the subject a first fluid composition that comprises the multi-arm polymer and a second fluid composition that comprises the multifunctional compound. In some of these embodiments, the first fluid composition and the second fluid composition are delivered using a double barrel syringe.

In some embodiments, which can be used in conjunction with the preceding aspects and embodiments, the method comprises administering to the subject a first fluid composition that comprises the multi-arm polymer and the multifunctional compound and a second fluid composition that comprises an accelerant that accelerates formation of covalent crosslinks between the multi-arm polymer and the multifunctional compound. In some of these embodiments, the first fluid composition and the second fluid composition are delivered using a double barrel syringe.

In other aspects, the present disclosure pertains to crosslinked hydrogel compositions comprising a crosslinked reaction product of (a) a multi-arm polymer in accordance with any of the above aspects and embodiments and (b) a multifunctional compound comprising a plurality of reactive groups that are reactive with reactive moieties of the multi-arm polymer, the reactive groups forming covalent bonds with the reactive moieties.

The multi-arm polymers of the present disclosure are advantageous relative to various prior polymers in that crosslinking is more controllable due to the new crosslinking reactions that they participate in.

The above and other aspects, embodiments, features and benefits of the present disclosure will be readily apparent from the following detailed description.

### BRIEF DESCRIPTION OF THE DRAWINGS

FIG. 1A schematically illustrates a reaction scheme for forming a hydroxyl-terminated multi-arm polymer with an iodinated core, in accordance with an embodiment of the present disclosure.

FIG. 1B schematically illustrates two additional reaction schemes for forming hydroxyl-terminated multi-arm polymers with iodinated cores, in accordance with further embodiments of the present disclosure.

FIG. 1C schematically illustrates a reaction scheme for forming a hydroxyl-terminated multi-arm polymer with an iodinated core, in accordance with yet another embodiment of the present disclosure.

FIG. 1D schematically illustrates a general reaction scheme for forming a multi-arm polymer having an iodinated core and reactive alkyl iodide end groups, a multi-arm polymer having an iodinated core and reactive vinyl ketone end groups, and a multi-arm polymer having an iodinated core and reactive ethynyl ketone end groups, in accordance with three embodiments of the present disclosure.

FIG. 1E schematically illustrates a reaction scheme for forming a hydrogel from (a) a multi-arm polymer having an iodinated core and a reactive moiety positioned at an end of each polymer arm of the multi-arm polymer and (b) a multifunctional compound comprising a plurality of reactive groups that are reactive with the reactive moieties of the multi-arm polymer, in accordance with another embodiment of the present disclosure.

FIG. 2 schematically illustrates a delivery device, in accordance with an embodiment of the present disclosure.

FIG. 3 schematically illustrates a delivery device, in accordance with another embodiment of the present disclosure.

### DETAILED DESCRIPTION

In some aspects, the present disclosure provides multi-arm polymers that comprise an iodinated core region and three or more polymer arms extending from the iodinated core region, each polymer arm of the three or more polymer arms comprising a hydrophilic polymer segment, and a reactive moiety. The reactive moiety is positioned at or near an end of each polymer arm and is covalently attached to the hydrophilic polymer segment.

The reactive moiety may be covalently attached to the hydrophilic polymer segment directly or via a variety of linkages that comprise ester groups, alkyl groups, ether groups, amide groups, amine groups, carbonate groups, and combinations of any of the foregoing, among others. In some embodiments, the reactive moiety is covalently attached to the hydrophilic polymer segment through a linkage that comprises a hydrolysable ester group,

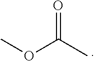

In some embodiments, the multi-arm polymers are hydrolysis resistant. In some embodiments, the reactive moiety may be covalently attached to the hydrophilic polymer segment through a hydrolysis resistant linkage, such as a linkage that comprises an alkyl group, an ether group, and amide group, or a combination of any of the foregoing.

In some embodiments, the reactive moiety comprises a C₁-C₁₀ alkyl iodide group or an amine group. In some embodiments, the reactive moiety comprises an α,β-unsaturated carbonyl group, for example, an α,β-unsaturated ketone group such as a vinyl ketone group

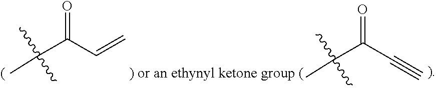

Multi-arm polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms (in other words, ranging between any two of the preceding values).

Hydrophilic polymer segments for the polymer arms of the multi-arm polymers of the present disclosure can be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Hydrophilic polymer segments for use in the multi-arm polymers of the present disclosure may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments and derivatives of the same, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Hydrophilic polymer segments for use in the multi-arm polymers of the present disclosure typically contain between 5 and 1000 monomer units or more, for example ranging from 5 to 10 to 25 to 50 to 100 to 250 to 500 to 1000 monomer units.

Iodinated core regions for use in the present disclosure include core regions that comprise a residue of an iodinated polyhydroxy compound comprising three or more hydroxyl groups, also referred to herein as an “iodinated polyol”, which is used to form the polymer arms. In certain beneficial embodiments, the core regions comprise residues of iodinated polyols that contain from 3 to 100 hydroxyl groups, for example, ranging anywhere from 3 to 4 to 5 to 6 to 7 to 8 to 9 to 10 to 11 to 12 to 15 to 20 to 25 to 50 to 75 to 100 hydroxy groups.

Illustrative iodinated polyols include iodinated aromatic polyols, examples of which are compounds that contain from 3 to 100 hydroxyl groups and one or more iodinated aromatic groups. Examples of iodinated aromatic groups include iodine-substituted monocyclic aromatic groups and iodine-substituted multicyclic aromatic groups, such as iodine-substituted benzene groups, iodine-substituted naphthalene groups, iodine-substituted anthracene groups, iodine-substituted phenanthrene groups and iodine-substituted tetracene groups, among others. The aromatic groups may be substituted with one, two, three, four, five, six or more iodine atoms. In various embodiments, the aromatic groups are further substituted with two or more hydroxyl-containing groups, including hydroxyl groups that are directly linked to the aromatic groups and hydroxyalkyl groups (e.g., C₁-C₄-hydroxyalkyl groups containing one, two, three or four carbon atoms and containing one, two, three or four or more hydroxyl groups), which may be linked to the aromatic group directly or through any suitable linking moiety, which may be selected, for example, from amide groups, ester groups, ether groups, carbamate groups, and combinations thereof, among others. Several specific iodinated polyols, along with the CAS numbers of the polyols and the number of hydroxyl groups in the polyols, are listed in the following table:

| | | # of |
|---|---|---|
| | | —OH |
| Compound | CAS # | Groups |
| 1,3,5-triiodo-2,4,6-tris-hydroxymethylbenzene | 126688-84-2 | 3 |
| Iopromide | 73334-07-3 | 4 |
| 5-Acetamido-N,N′-bis(2,3-dihydroxypropyl)- | 31127-80-7 | 4 |
| 2,4,6-triiodoisophthalamide | | |
| Metrizamide | 31112-62-6 | 4 |
| 1,3-Benzenedicarboxamide,5-(acetylamino)- | 76350-28-2 | 4 |
| N1,N3-bis(2,3-dihydroxypropyl)-2,4,6- | | |
| triiodo-N1-methyl-(ACI) | | |
| Iopamidol | 60166-93-0 | 5 |
| Iomeprol | 78649-41-9 | 5 |
| 1,3-Benzenedicarboxamide,N1,N3-bis[2-hydroxy- | 60208-45-9 | 5 |
| 1-(hydroxymethyl)ethyl]-5-[(2-hydroxy-1- | | |
| oxopropyl)amino]-2,4,6-triiodo-(ACI) | | |
| Ioxilan | 107793-72-6 | 5 |
| Iopentol | 89797-00-2 | 5 |
| Ioversol | 87771-40-2 | 6 |
| Iohexol | 66108-95-0 | 6 |
| Iobitridol | 136949-58-1 | 6 |
| Iodixanol | 92339-11-2 | 9 |
| 1,3-Benzenedicarboxamide,5,5′-[(1,3-dioxo-1,3- | 79770-24-4 | 12 |
| propanediyl)bis(methylimino)]bis[N,N′-bis[2,3- | | |
| dihydroxy-1-(hydroxymethyl)propyl]-2,4,6- | | |
| triiodo-(9CI, ACI) | | |

Further various iodinated polyols can be found, for example, in U.S. Ser. No. 18/762,216, the disclosure of which is hereby incorporated by reference. These include various iodinated polyols that contain 8 hydroxyl groups and contain four 1,3,5-triiodo-2,4-dihydroxymethyl benzene groups such as the following:

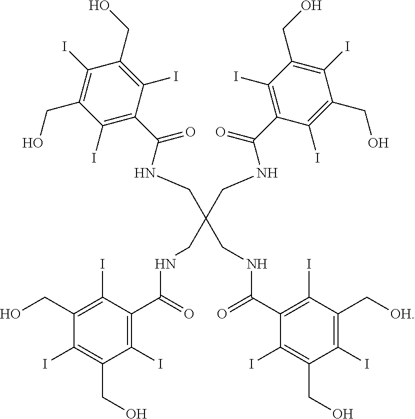 [SMILES: O=C(NCC(CNC(=O)c1c(I)c(CO)c(I)c(CO)c1I)(CNC(=O)c1c(I)c(CO)c(I)c(CO)c1I)CNC(=O)c1c(I)c(CO)c(I)c(CO)c1I)c1c(I)c(CO)c(I)c(CO)c1I]

among others.

In some embodiments of the present disclosure, an iodinated polyol such as one of those described above, among others, may be used as a multi-functional initiator for polymer chain growth.

For example, in some embodiments, an iodinated polyol may be used as an initiator for ring-opening polymerization of ethylene oxide to form polyethylene oxide (PEO) segments (also referred to as polyethylene glycol (PEG) segments) at a position of each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity in some embodiments.

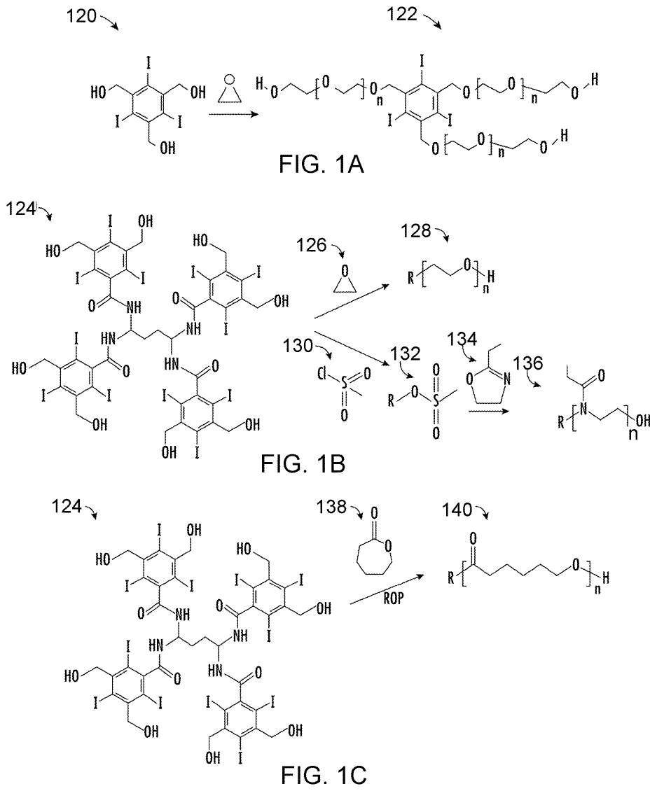

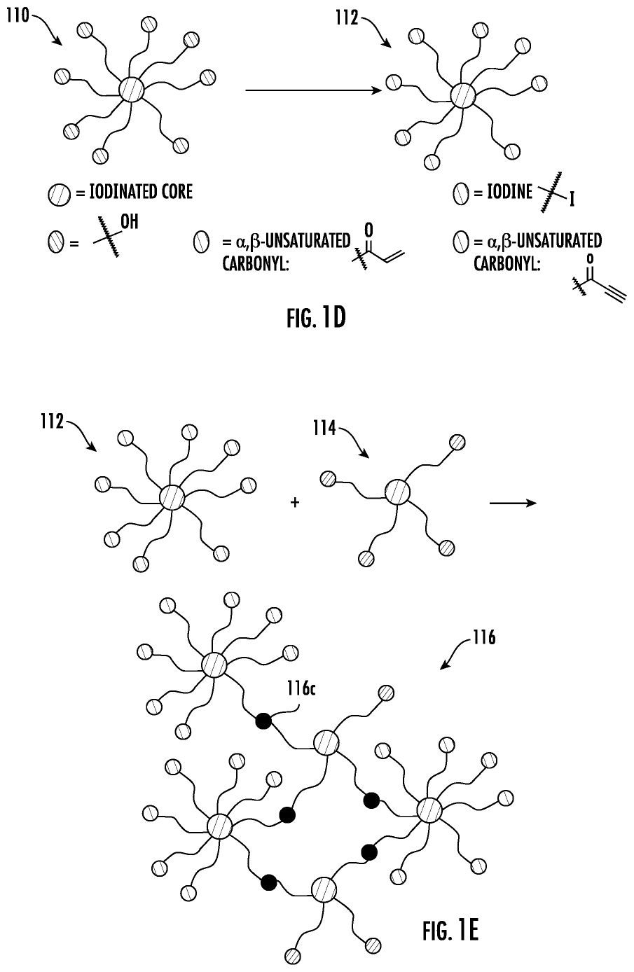

In a particular example shown in FIG. 1A, an iodinated polyol, 1,3,5-triiodo-2,4,6-tris-hydroxymethylbenzene 120, may be used to initiate a ring-opening polymerization step with ethylene oxide. The polymerization process leads to poly(ethylene oxide) chain growth at each of the three hydroxyl groups of the iodinated polyol, forming an iodinated, multi-arm polymer 122 that contains three poly(ethylene oxide) arms, where n is an integer and may range, for example, from 5 to 500, which poly(ethylene oxide) arms extend from a 1,3,5-triiodo-2,4,6-tris-hydroxymethylbenzene residue core. Each of the poly(ethylene oxide) arms has a terminal hydroxyl group.

In another particular example shown in FIG. 1B, an iodinated polyol 124 may be used to initiate a ring-opening polymerization step with ethylene oxide 126. The polymerization process leads to poly(ethylene oxide) chain growth at each of the eight hydroxyl groups of the iodinated polyol 124, forming an iodinated, multi-arm polymer 128 having eight hydroxy-terminated poly(ethylene oxide) arms where n ranges from 5 to 500. It is noted that only one of the eight hydroxy-terminated poly(ethylene oxide) arms is illustrated, and that R represents a residue of the iodinated polyol 124 and the remaining seven polymer arms.

Analogous methods can be performed with many other iodinated polyols including those described above. For example, a hydroxy-terminated polymer eight-arm polymer,

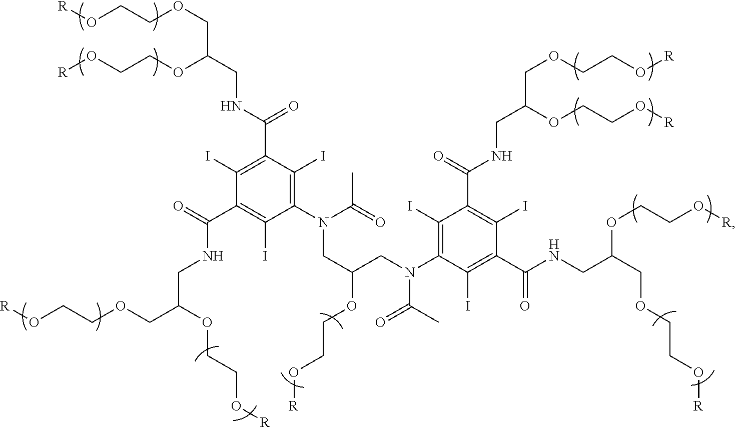

where R is a hydroxyl group, can be formed using iodixanol as the iodinated polyol, where the parentheses designate polyethylene oxide segments, which can contain, for example, from 5 to 500 ethylene oxide units per arm.

As another example, an iodinated polyol may be reacted with methanesulfonyl chloride to convert hydroxyl groups of the iodinated polyol into methanesulfonate groups, which can serve as leaving groups for the cationic polymerization of an oxazoline monomer to form polyoxazoline segments at a site of each of the hydroxyl groups of the polyol. The resulting hydroxyl-terminated polyoxazoline segments possess tunable hydrophilicity depending on the desired water-solubility of the resulting multi-arm polymer, for example, with increasing polyoxazoline segment length leading to increased hydrophilicity in some embodiments.

In s particular embodiment shown in FIG. 1B, an iodinated polyol 124 can be reacted with methanesulfonyl chloride 130, to form a multifunctional initiator molecule 132 comprising a plurality of sulfonyl polymerization groups in the place of the hydroxyl groups of the iodinated polyol 124 (only one of the eight methanesulfonyl groups is illustrated and R represents a remainder of the multifunctional initiator molecule 132). The multifunctional initiator molecule 132 is then used to create a radiopaque, multi-arm polyoxazoline via ring opening polymerization of one or more oxazoline monomers, specifically 2-ethyl-2-oxazoline 134, with the formation of an iodinated, multi-arm polymer 136 having eight hydroxy-terminated poly(2-ethyl-2-oxazoline) arms, where n ranges from 5 to 500. It is noted that only one of the eight hydroxy-terminated poly(2-ethyl-2-oxazoline) arms is illustrated, with a residue of the iodinated polyol 124 and the remaining seven hydroxyl-terminated poly(2-ethyl-2-oxazoline) polymer arms represented by R. Other sulfonic acid chlorides, such as trifluoromethanesulfonyl chloride or tosyl chloride may be employed as well. Analogous methods can be performed with other iodinated polyols including those described above.

As further example, and with reference to FIG. 1C, an iodinated polyol 124 is used to initiate a ring-opening polymerization step with ε-caprolactone 138 to form an iodinated, multi-arm polymer 140 having eight hydroxy-terminated polyester arms (i.e., poly(ε-caprolactone) arms), where n ranges from 1 to 150. It is noted that only one of the eight hydroxy-terminated poly(ε-caprolactone) arms is illustrated, with R representing a residue of the iodinated polyol 124 and the remaining seven hydroxy-terminated poly(2-ethyl-2-oxazoline arms. Analogous methods can be performed with other iodinated polyols including those described above.

Further examples of polymer chain growth from iodinated polyols can be found, for example, in U.S. Ser. Nos. 18/762,216 and 18/979,225, the disclosures of which are hereby incorporated by reference.

Using these and other strategies, a variety of multi-arm polymers having a variety of iodinated polyol residue cores and a variety of hydroxy-terminated polymer arms may be provided. Such hydroxy-terminated multi-arm polymers can be used to form multi-arm polymers in accordance with the present disclosure through various synthetic routes.

For instance, as previously noted, in some embodiments, the multi-arm polymers of the present disclosure comprise reactive moieties that comprise C₁-C₁₀ alkyl iodide groups. Such multi-arm polymers can be formed from hydroxy-terminated multi-arm polymers such as those described herein. As a particular example, such multi-arm polymers can be formed based on classic iodination synthesis from hydroxyl alkanes. In this regard, by reacting a hydroxy-terminated multi-arm polymer (e.g., hydroxy-terminated multi-arm PEG, among many among others) with a mixture of sodium or potassium iodide and concentrated phosphoric(V) acid (H₃PO₄), hydrogen iodide (HI) is produced, which then reacts with the hydroxyl group to form an iodine substituent, yielding a multi-arm C₁-C₁₀-alkyl-iodide-terminated polymer as schematically shown here (when n ranges from 5 to 500):

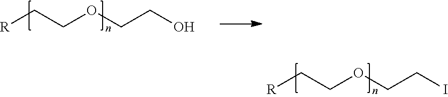

As also previously noted, in some embodiments, the multi-arm polymers of the present disclosure comprise reactive moieties that comprise an α,β-unsaturated carbonyl moiety. Such multi-arm polymers can be formed from hydroxy-terminated multi-arm polymers such as those described herein. As a particular example, a multi-arm C₁-C₁₀-alkyl-iodide-terminated polymer such as that formed in the prior paragraph can be reacted in a normal SN2 reaction with a hydroxyl-tailing α,β-unsaturated carbonyl compound, for example, a hydroxyl-1-C₄-C₁₀-alken-3-one compound, such as 4-hydroxyl-1-buten-3-one, 5-hydroxyl-1-penten-3-one, 6-hydroxyl-1-hexen-3-one, 7-hydroxyl-1-hepten-3-one, and 8-hydroxyl-1-octen-3-one, among others, in the presence of K₂CO₃ in acetone or other polar aprotic solvent at slightly elevated temperature, as schematically shown here with 7-hydroxyl-1-hepten-3-one (when n ranges from 5 to 500):

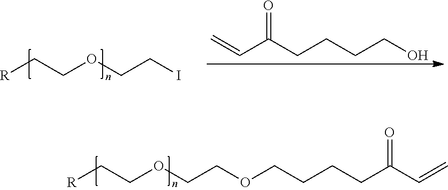

While this reaction produces vinyl ketone groups, analogous reactions can be employed to produce ethynyl ketone groups using hydroxyl-1-C₄-C₁₀-alkyn-3-one compounds such as 4-hydroxyl-1-butyn-3-one, 5-hydroxyl-1-pentyn-3-one, 6-hydroxyl-1-hexyn-3-one, 7-hydroxyl-1-heptyn-3-one, and 8-hydroxyl-1-octyn-3-one, among others.

Embodiments of such processes are shown schematically in FIG. 1D where a hydroxyl-terminated 8-arm polymer 110 with an iodinated core is used to form an 8-arm polymer 112 with an iodinated core and polymer arms having reactive alkyl iodide end groups, reactive vinyl ketone end groups, or reactive ethynyl ketone end groups.

As previously indicated, multi-arm polymers in accordance with the present disclosure include those with polymer arms in which a reactive moiety is linked to a hydrophilic polymer segment through a linkage that comprises an ester group. In some of these embodiments, the ester linkage is formed from a cyclic anhydride compound, and the reactive moiety is covalently linked to the hydrophilic polymer segment through a residue of the cyclic anhydride compound. In these embodiments the multi-arm polymers have polymer arms in which a cyclic anhydride residue is covalently linked to a hydrophilic polymer segment and in which a reactive moiety is covalently linked to the cyclic anhydride residue.

Cyclic anhydrides may be reacted with a hydroxy-terminated multi-arm polymer such as is described above under basic conditions to form an iodinated carboxylic-acid-terminated precursor polymer comprising a carboxylic acid end group that is linked to a hydrophilic polymer segment through a hydrolysable ester group. A particular example is schematically shown here (wherein n ranges from 5 to 500) using glutaric anhydride as the cyclic anhydride:

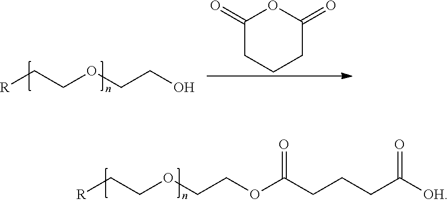

A reactive moiety, for example, a reactive moiety that comprises a C₁-C₁₀ alkyl iodide group or an α,β-unsaturated ketone group, may then be linked to the iodinated carboxylic-acid-terminated precursor polymer.

In various embodiments, hydroxyl groups of the iodinated hydroxy-terminated multi-arm polymer are reacted in a ring-opening reaction with an iodinated or non-iodinated cyclic anhydride (e.g., glutaric anhydride, succinic anhydride, malonic anhydride, adipic anhydride, diglycolic anhydride, etc.) to form a reaction product that comprises carboxylic acid end groups Various anhydrides, along with their CAS numbers, some of which are iodinated, are listed in the following table.

| Species | CAS # |
|---|---|
| Succinic Anhydride | 108-30-5 |
| Glutaric Anhydride | 108-55-4 |
| Adipic Anhydride | 2035-75-8 |
| 2,8-Oxocanedione | 10521-07-0 |
| 3-Oxabicyclo[3.1.0]hexane-2,4-dione | 5617-74-3 |
| 3-Oxabicyclo[3.2.0]heptane-2,4-dione | 4462-96-8 |
| Dihydro-4-methyl-2H-pyran-2,6(3H)-dione | 4166-53-4 |
| Itaconic anhydride | 2170-03-8 |
| 2,2-Dimethylsuccinic anhydride | 17347-61-4 |
| 6,6-Dimethyl-3-oxabicyclo[3.1.0]hexane-2,4-dione | 5466-84-2 |
| Dihydro-4,4-dimethyl-2H-pyran-2,6(3H)-dione | 4160-82-1 |
| Diglycolic anhydride | 4480-83-5 |
| 2H-Pyran-2,4,6(3H,5H)-trione | 10521-08-1 |
| Dihydro-4-(2-methylpropyl)-2H-pyran-2,6(3H)-dione | 185815-59-2 |
| 3-Iodophthalic anhydride* | 28418-88-4 |
| Tetraiodophthalic anhydride* | 632-80-4 |
| 5-Iodo-1,3-isobenzofurandione* | 28418-89-5 |
| 5,6-Diiodo-1,3-isobenzofurandione* | 25834-17-7 |
| Dihydro-3-(2-iodophenyl)-2,5-furandione* | 887131-98-8 |

*Iodinated Compounds.

A reactive moiety may be linked to the carboxyl groups of the iodinated carboxyl-terminated multi-arm polymer that is produced by the above-described ring-opening reaction through various synthetic routes.

Instance, as previously noted, in some embodiments, the multi-arm polymers of the present disclosure comprise reactive moieties that comprise C₁-C₁₀ alkyl iodide groups. Such multi-arm polymers can be formed from carboxyl-terminated multi-arm polymers such as those described herein. As a particular example, such multi-arm polymers can be formed based on ester coupling between carboxyl group of a carboxyl-terminated multi-arm polymer and a hydroxyl group of an iodo-C₁-C₁₀-alkanol (e.g., iodomethanol, 2-iodo-1-ethanol, 3-iodo-1-propanol, 4-iodo-1-butanol, 5-iodo-1-pentanol, 6-iodo-1-hexanol, 7-iodo-1-heptanol, 8-iodo-1-octanol, etc.) in the presence of a suitable coupling agent such as a carbodiimide coupling agent.

As also previously noted, in some embodiments, the multi-arm polymers of the present disclosure comprise reactive moieties that comprise α,β-unsaturated carbonyl groups. Such multi-arm polymers can be formed from carboxyl-terminated multi-arm polymers such as those described herein. As a particular example, such multi-arm polymers can be formed based on ester coupling between the carboxyl groups of a carboxyl-terminated multi-arm polymer and a hydroxyl group of a hydroxyl tailing α,β-unsaturated carbonyl compound in the presence of a suitable coupling agent such as a carbodiimide coupling agent. Examples of hydroxyl tailing α,β-unsaturated carbonyl compounds include, for example, hydroxyl-1-C₄-C₁₀-alken-3-one compounds, such as 4-hydroxyl-1-buten-3-one, 5-hydroxyl-1-penten-3-one, 6-hydroxyl-1-hexen-3-one, 7-hydroxyl-1-hepten-3-one, and 8-hydroxyl-1-octen-3-one, among others, as schematically shown here with 7-hydroxyl-1-hepten-3-one (when n ranges from 5 to 500):

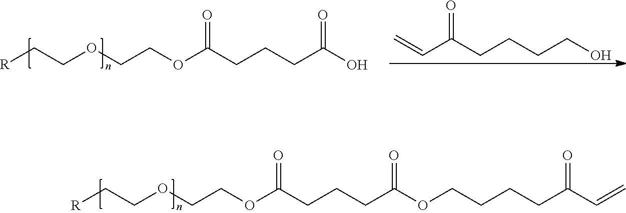

While this reaction produces a vinyl ketone groups, analogous reactions can be employed to produce ethynyl ketone groups using hydroxyl-1-C₄-C₁₀-alkyn-3-one compounds such as 4-hydroxyl-1-butyn-3-one, 5-hydroxyl-1-pentyn-3-one, 6-hydroxyl-1-hexyn-3-one, 7-hydroxyl-1-heptyn-3-one, and 8-hydroxyl-1-octyn-3-one, among others.

In other aspects, the present disclosure provides systems that comprise (a) a multi-arm polymer as described above, which comprises an iodinated core region and three or more polymer arms extending from the iodinated core region, each polymer arm of the three or more polymer arms comprising a hydrophilic polymer segment, and a reactive moiety, and (b) a multifunctional compound comprising a plurality of reactive groups that are reactive with the reactive moieties of the multi-arm polymer.

In some embodiments, the multifunctional compound is a compound having at two or more reactive groups, for example, having 2, 3, 4, 5, 6, 7, 8, 9, 10, 11, 12, 13, 14, 15, 16, 17, 18, 19, 20 or more second reactive groups. Suitable reactive groups include nucleophilic groups. Specific nucleophilic groups include amino groups and thiol groups.

Where the reactive moieties of the multi-arm polymer comprise an α,β-unsaturated carbonyl group, the reactive groups of the multifunctional compound may be nucleophilic groups. In this regard, α,β-unsaturated carbonyl groups react with nucleophilic groups, primarily through a mechanism called 1,4-addition, or Michael addition, where the nucleophile adds to the beta-carbon. For example, a α,β-unsaturated carbonyl group can react with a thiol group through a 1,4-addition reaction to form a new sulfur-carbon bond at the position of the beta-carbon. As another example, an α,β-unsaturated carbonyl group can react with a primary or secondary amine group through a 1,4-addition reaction to form a new nitrogen-carbon bond at the position of the beta-carbon.

Where the reactive moieties of the multi-arm polymer comprise an alkyl iodide group, the reactive groups of the of the multifunctional compound may be nucleophilic groups. Alkyl iodide groups react with nucleophilic groups primarily through a nucleophilic substitution reaction, forming new bonds between the alkyl portion of the alkyl iodide group and the nucleophile. For example, an alkyl iodide group can react in a nucleophilic substitution reaction with a thiol group to form a new sulfur-carbon bond at the site formerly occupied by the iodine atom of the alkyl iodide group. As another example, an alkyl iodide group can react in a nucleophilic substitution reaction with a primary or secondary amine group to form a new nitrogen-carbon bond at the site formerly occupied by the iodine atom of the alkyl iodide group.

Some specific examples of multifunctional compounds include, for example, polyamines that contain two or more amino (—NH₂) groups (e.g., 2, 3, 4, 5, 6, 7, 8, 9, 10 or more amino groups in some embodiments), also referred to herein as polyamino compounds. Polyamino compounds suitable for use in the present disclosure include polyamino compounds that comprise two or more primary amine groups, for example, —(CH₂)ₓ—NH₂ groups where x is 1, 2, 3, 4, 5 or 6. Polyamino compounds suitable for use in the present disclosure include polyamino compounds that comprise two or more basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Further examples of polyamino compounds which may be used as the multifunctional compound include ethylenediamine, ethylenetriamine, diethylene triamine, hexamethylenetriiamine, di(heptamethylene) triamine, di(trimethylene) triamine, bis(hexamethylene) triamine, tris(2-aminoethyl)amine, tris(3-aminopropyl)amine, 1,3,5-tris-(2-aminoethyl)-[1,3,5]triazinane-2,4,6-trione, N,N,N′-tris(2-aminoethyl)ethylenediamine, triethylene tetramine, tripropylene tetramine, N,N′,N′-tetrakis(2-aminoethyl)-1,2-ethanediamine, tetraethylene pentamine, hexamethylene heptamine, pentaethylene hexamine, dimethyl octylamine, dimethyl decylamine, and JEFFAMINE polyetheramines available from Huntsman Corporation, and poly(allyl amine), among others.

Compounds having at least two amino groups may also be made from polyols such those described above. For example, hydroxyl groups of a polyol maybe reacted in an ester coupling reaction in the presence of a suitable coupling agent with a carboxyl group of a suitable amino acid compound in which the amino group of the amino acid is protected with a suitable protective group (e.g., a tert-butyloxycarbonyl (tBoc) protective group). Examples of amino acids may be selected, for example, from beta amino acids such as 3-aminopropanoic acid (also known as beta-alanine), gamma amino acids such as 4-aminobutanoic acid (also known as gamma-aminobutyric acid, or GABA), delta amino acids such as 5-aminopentanoic acid, epsilon amino acids such as 6-aminohexanoic acid, etc. Deprotection of the protective groups results in a compound in which a primary amine group is linked to a residue of the polyol through a hydrolysable ester group at the site of each of the hydroxyl groups of the polyol.

Specific examples of compounds having at least two thiol groups include hydrophilic dithiols such as ethylene glycol bis(3-mercaptopropionate), 2,3-butanedithiol, 1,10-decanedithiol, dithioerythritol, ethylene bis(thioglycolate), 3,6-dioxa-1,8-octanedithiol, 3,7-dithia-1,9-nonanedithiol, ethylene glycol bis(3-mercaptopropionate), 1,6-hexanedithiol, bis(2-mercaptoethyl) sulfide, 1,3-propanedithiol, hexa(ethylene glycol) dithiol, 1,4-butanediol bis(thioglycolate), pentaerythritol tetrakis(mercaptoacetate), 1,5-pentanedithiol, dipentaerythritol hexakis(3-mercaptopropionate), pentaerythritol tetra(3-mercaptopropionate), trimethylolpropane tris(thioglycolate), and trimethylolpropane tris(3-mercaptopropionate), among others. Compounds having at least two thiol groups may also be made converting hydroxyl group of polyols such those described above to thiol groups using a nucleophilic substitution reaction with a sulfur-containing reagent, such as sodium hydrosulfide (NaSH).

In some aspects of the present disclosure, crosslinked hydrogels are provided that comprise (a) a multi-arm polymer as described above, which comprises a iodinated core region and three or more polymer arms extending from the iodinated core region, each polymer arm of the three or more polymer arms comprising a hydrophilic polymer segment and a reactive moiety and (b) a multifunctional compound comprising a plurality of reactive groups that are reactive with the reactive moieties of the multi-arm polymer.

As used herein, a “hydrogel” is a crosslinked polymer that contains water or can absorb water but does not dissolve when placed in water.

The hydrogels of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values) for example, when measured on a bench-top micro CT system such as XtremeCT from Scanco Medical (Wangen-Brüttisellen, Switzerland) or similar.

The hydrogels of the present disclosure can be used in a variety of biomedical applications, including implants, medical devices, and pharmaceutical compositions.

In various aspects of the present disclosure, systems are provided that comprise (a) a first composition that comprises a multi-arm polymer as described herein and (b) a second composition that comprises multifunctional compound as described herein, wherein when the first and second compositions are combined, covalent crosslinks form between the multi-arm polymer and the multifunctional compound, creating a hydrogel.

This process is shown schematically in FIG. 1E, wherein an 8-arm polymer 112 like that of FIG. 1D, which has an iodinated core and polymer arms having reactive α,β-unsaturated ketone groups, is reacted with a multifunctional compound 114 having four nucleophilic groups, for example, trilysine, which contains four primary amine groups. The reactive alkyl iodide end groups, reactive vinyl ketone end groups or reactive α,β-unsaturated ketone groups of the multi-arm polymer 112 react with the nucleophilic groups of the multifunctional compound 114 to form covalent bonds 116c that form a hydrogel 116.

The first composition may be a first fluid composition comprising the multi-arm polymer or a first dry composition that comprises the multi-arm polymer, to which a suitable fluid such as water for injection, saline, etc. can be added to form a first fluid composition. In addition to the multi-arm polymer, the first composition may further comprise additional agents, including therapeutic agents, imaging agents, colorants, tonicity adjusting agents, suspension agents, wetting agents, and pH adjusting agents as described below.

The second composition may be a second fluid composition comprising the multifunctional compound or a second dry composition that comprises the multifunctional compound, to which a suitable fluid such as water for injection, saline, etc. can be added to form a second fluid composition. In addition to the multifunctional compound, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the multi-arm polymer with a second fluid comprising the multifunctional compound, and the first composition, the second composition, or both, may optionally comprise additional agents as described herein. Upon mixing the first and second fluid compositions, the multifunctional compound forms crosslinks with the multi-arm polymer, forming a hydrogel. The first and second fluid compositions may be combined to form crosslinked hydrogels, either in vivo or ex vivo.

In some embodiments, the multi-arm polymer is initially combined with the multifunctional compound under conditions where crosslinking between the multi-arm polymer and the multifunctional compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the multi-arm polymer and the multifunctional compound, thereby forming a crosslinked product.

In some embodiments, the system comprises (a) a first composition that comprises a multi-arm polymer as described herein and a multifunctional compound as described herein and (b) a second composition, specifically, an accelerant composition, that contains an accelerant that is configured to accelerate a crosslinking reaction between the multifunctional compound and the multi-arm polymer. In some embodiments, the first composition may further comprise additional agents as described herein. In some embodiments, the second composition may further comprise additional agents as described herein.

In some embodiments, the system comprises (a) a first composition that comprises multifunctional compound as described herein, (b) a second composition that comprises a multi-arm polymer as described herein, and (c) a third composition, specifically, an accelerant composition, that contains an accelerant that is configured to accelerate a crosslinking reaction between the multifunctional compound and the multi-arm polymer. In some embodiments, the first composition may further comprise additional agents as described herein. In some embodiments, the second composition may further comprise additional agents as described herein. In some embodiments, the third composition may further comprise additional agents as described herein.

The first composition may be a first fluid composition comprising the multifunctional compound that is buffered to an acidic pH or a first dry composition that comprises the multifunctional compound, to which a suitable fluid such as water for injection, saline, an acidic buffer solution, etc. can be added to form a first fluid composition comprising the multifunctional compound that is buffered to an acidic pH. In some embodiments, for example, the acidic buffering composition may comprise monobasic sodium phosphate, among other possibilities. The first fluid composition comprising the multifunctional compound may have a pH ranging, for example, from about 3 to about 5. In addition to the multifunctional compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the multi-arm polymer or a second dry composition that comprises the multi-arm polymer from which a fluid composition is formed, for example, by the addition of a suitable fluid such as water for injection, saline, or the first fluid composition comprising the multifunctional compound that is buffered to an acidic pH. In addition to the multi-arm polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the multifunctional compound (as well as additional agents in some cases) that is buffered to an acidic pH and the second composition is a dry composition that comprises the multi-arm polymer (as well as additional agents in some cases). The first composition may then be mixed with the second composition to provide a prepared fluid composition that is buffered to an acidic pH and comprises the multifunctional compound and the multi-arm polymer. In a particular example, a syringe may be provided that contains the first fluid composition comprising the multifunctional compound that is buffered to an acidic pH, and a vial may be provided that comprises the second dry composition (e.g., a powder) that comprises the multi-arm polymer. The syringe may then be used to inject the first fluid composition into the vial containing the multi-arm polymer to form a prepared fluid composition that is buffered to an acidic pH and contains the multifunctional compound and the multi-arm polymer, which can be withdrawn back into the syringe for administration.

The accelerant composition may be a fluid accelerant composition that is buffered to a basic pH or a dry composition that comprises a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the multifunctional compound and the multi-arm polymer as described above (as well as additional agents in some cases) and a fluid accelerant composition that is buffered to basic pH as described above (which may include additional agents in some cases) may be combined to form crosslinked hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, and (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others.

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises a multi-arm polymer as described above and a second reservoir that contains a second fluid composition that comprises a multifunctional compound as described above, wherein the first and second fluid compositions form a crosslinked product upon mixing. As previously noted, the first fluid composition, the second fluid composition, or both, may optionally comprise additional agents as described herein.

In some embodiments, the system may include a delivery device that comprises a first reservoir that contains a first fluid composition that comprises the multifunctional compound and the multi-arm polymer and is buffered to an acidic pH, such as the prepared fluid composition previously described, and a second reservoir that contains second fluid composition, such as the fluid accelerant composition previously described. As previously noted, the first fluid composition, the second fluid composition, or both, may optionally comprise additional agents as described herein.

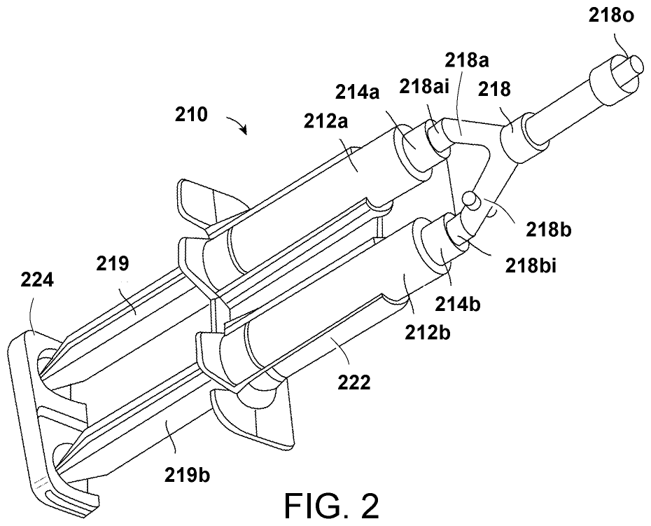

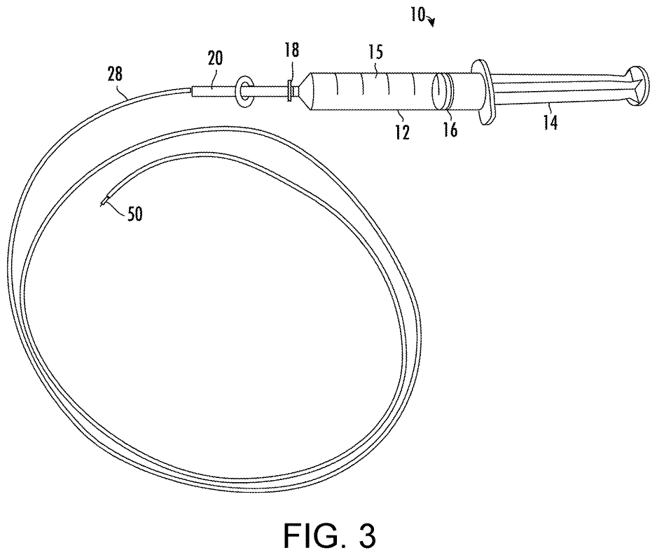

In either case, during operation, the first fluid composition and second fluid composition are dispensed from the first and second reservoirs and combined, whereupon the multifunctional compound and the multi-arm polymer and crosslink with one another to form a crosslinked hydrogel. In particular embodiments, and with reference to FIG. 2, the system may include a delivery device 210 that comprises a double-barrel syringe, which includes a first barrel 212a having a first barrel outlet 214a, which first barrel contains a first fluid composition as described above, a first plunger 219a that is movable in the first barrel 212a, a second barrel 212b having a second barrel outlet 214b, which second barrel 212b contains a second fluid composition as described above, and a second plunger 219b that is movable in the second barrel 212b. In some embodiments, the device 210 may further comprise a mixing section 218 having a first mixing section inlet 218ai in fluid communication with the first barrel outlet 214a, a second mixing section inlet 218bi in fluid communication with the second barrel outlet 214b, and a mixing section outlet 218o. Also shown are a syringe holder 222 configured to hold the first and second syringe barrels 212a, 212b, in a fixed relationship and a plunger cap 224 configured to hold the first and second plungers 219a, 219b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

In some embodiments, the delivery device may further comprise a needle or catheter tube that is configured to receive the first and second fluid compositions from the first and second barrels. For example, a needle, cannula or catheter tube may be configured to form a fluid connection with an outlet of a mixing section by attaching the needle, cannula or catheter tube to an outlet of the mixing section, for example, via a suitable fluid connector such as a Luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, needle, cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

During and/or after administration, the compositions of the present disclosure can be imaged using a suitable imaging technique. Typically, the imaging technique is an x-ray-based imaging technique, such as computerized tomography or X-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may vary widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a cannula; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

FIG. 14 illustrates a syringe 10 providing a reservoir for a hydrogel composition in accordance with the present disclosure. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

During and/or after administration, the crosslinked hydrogel compositions of the present disclosure can be imaged using a suitable imaging technique.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A multi-arm polymer comprising an iodinated core region and three or more polymer arms extending from the iodinated core region, each polymer arm of the three or more polymer arms comprising a hydrophilic polymer segment and a reactive moiety, wherein the reactive moiety is positioned at an end of each polymer arm and is covalently attached to the hydrophilic polymer segment, and wherein the reactive moiety comprises a C₁-C₁₀ alkyl iodide group or an α,β-unsaturated ketone group.

2. The multi-arm polymer of claim 1, wherein the reactive moiety comprises the C₁-C₁₀ alkyl iodide group.

3. The multi-arm polymer of claim 1, wherein the reactive moiety comprises the α,β-unsaturated ketone group.

4. The multi-arm polymer of claim 3, wherein the α,β-unsaturated ketone group is a vinyl ketone group.

5. The multi-arm polymer of claim 3, wherein the α,β-unsaturated ketone group is an ethynyl ketone group.

6. The multi-arm polymer of claim 1, wherein the reactive moiety is covalently linked to the hydrophilic polymer segment through a linkage that comprises an ester group.

7. The multi-arm polymer of claim 1, wherein the reactive moiety is covalently linked to the hydrophilic polymer segment through a hydrolytically stable linkage.

8. The multi-arm polymer of claim 1, wherein the hydrophilic polymer segment comprises one or more monomer residues selected from ethylene oxide, propylene oxide, N-vinyl pyrrolidone and oxazoline monomer residues.

9. The multi-arm polymer of claim 1, wherein the iodinated core comprises one or more iodinated aromatic groups.

10. The multi-arm polymer of claim 1, wherein the iodinated core region comprises a residue of an iodinated polyhydroxy compound.

11. A system comprising (a) a multi-arm polymer comprising an iodinated core region and three or more polymer arms extending from the iodinated core region, each polymer arm of the three or more polymer arms comprising a hydrophilic polymer segment and a reactive moiety, wherein the reactive moiety is positioned at an end of each polymer arm and is covalently attached to the hydrophilic polymer segment, and wherein the reactive moiety comprises a C₁-C₁₀ alkyl iodide group or an α,β-unsaturated ketone group and (b) a multifunctional compound comprising a plurality of reactive groups that are reactive with reactive moieties of the multi-arm polymer.

12. The system of claim 11, wherein the reactive groups are nucleophilic groups.

13. The system of claim 12, wherein the nucleophilic groups are primary amine groups or secondary amine groups.

14. The system of claim 12, wherein the nucleophilic groups are thiol groups.

15. A method of treatment comprising administering to a subject a mixture that comprises (a) a multi-arm polymer comprising an iodinated core region and three or more polymer arms extending from the iodinated core region, each polymer arm of the three or more polymer arms comprising a hydrophilic polymer segment and a reactive moiety, wherein the reactive moiety is positioned at an end of each polymer arm and is covalently attached to the hydrophilic polymer segment, and wherein the reactive moiety comprises a C₁-C₁₀ alkyl iodide group or an α,β-unsaturated ketone group and (b) a multifunctional compound comprising a plurality of reactive groups that are reactive with reactive moieties of the multi-arm polymer, under conditions such that the multi-arm polymer and the multifunctional compound crosslink after administration to form a hydrogel.

16. The method of claim 15, wherein the nucleophilic groups are selected from thiol groups, primary amine groups and secondary amine groups.

17. The method of claim 15, wherein the method comprises administering to the subject a first fluid composition that comprises the multi-arm polymer and a second fluid composition that comprises the multifunctional compound.

18. The method of claim 15, wherein the method comprises administering to the subject a first fluid composition that comprises the multi-arm polymer and the multifunctional compound and a second fluid composition that comprises an accelerant that accelerates formation of covalent crosslinks between the multi-arm polymer and the multifunctional compound.

19. The method of claim 18, wherein the first fluid composition and the second fluid composition are delivered using a double barrel syringe.

20. A crosslinked hydrogel composition comprising a crosslinked reaction product of (a) the multi-arm polymer of claim 1 and (b) a multifunctional compound comprising a plurality of reactive groups that are reactive with reactive moieties of the multi-arm polymer, the reactive groups forming covalent bonds with the reactive moieties.
